(19) United States
(12) Patent Application Publication
(10) Pub. No.: US 2020/0051191 A1
(43) Pub. Date: Feb. 13, 2020

(54) SYSTEMS AND METHODS FOR PROVIDING FINANCIAL ANALYSES FOR SERVICE PROVIDERS

(71) Applicant: CSF BUSINESS CONSULTING, LLC, Bountiful, UT (US)

(72) Inventor: Christopher Tayler, Bountiful, UT (US)

(73) Assignee: CSF BUSINESS CONSULTING, LLC, Bountiful, UT (US)

(21) Appl. No.: 16/657,934

(22) Filed: Oct. 18, 2019

Related U.S. Application Data
(63) Continuation of application No. 13/931,378, filed on Jun. 28, 2013, now abandoned.
(60) Provisional application No. 61/665,823, filed on Jun. 28, 2012.

(51) Int. Cl. G06Q 50/22; G06Q 40/00; G06Q 10/06

(52) U.S. Cl. CPC G06Q 50/22; G06Q 10/0637; G06Q 40/12; G06Q 40/10

(57) ABSTRACT

The present invention relates to systems and methods for providing financial analyses to service providers. In some cases, the inventions relates to systems that gathers information, for a chosen period of time, about a subset of chosen procedures selected from all procedures performed by a service provider's practice. This gathered information can include, among other things, a time required for the provider to perform each of the chosen procedures, a fee for service charge the provider charges for each chosen procedure; a fee an insurance company is willing to pay for each of the chosen procedures; and a number of each of the chosen procedures the provider performed in the chosen period. In some case, the systems determine a weighted profit/loss for each of the chosen procedures under the fee for service charge and under fee that the insurance company is willing to pay.

### RELATED APPLICATIONS

This is a continuation application which claims priority to U.S. patent application Ser. No. 13/931,378, filed Jun. 28, 2013, entitled SYSTEMS AND METHODS FOR PROVIDING FINANCIAL ANALYSES FOR SERVICE PROVIDERS (Attorney Docket No. 20246.3), which claims priority to U.S. Provisional Patent Application Ser. No. 61/665,823, filed Jun. 28, 2012, entitled SYSTEMS AND METHODS FOR PROVIDING FINANCIAL ANALYSES FOR SERVICE PROVIDERS (Attorney Docket No. 20246.2); both of which are incorporated herein by reference in their entirety.

### BACKGROUND OF THE INVENTION

### Field of the Invention

The present invention relates to systems and methods for providing financial analyses to service providers. In particular, some implementations of the present invention relate to systems and methods for determining the profitability or loss associated with certain procedures and certain insurance companies.

### Background and Related Art

For many service providers, such as those in the healthcare industry, the cost of performing procedures can increase on a regular basis. For instance, the costs associated with tools, supplies, employees, etc. may increase and, thereby, reduce overall the amount of profit that is earned from each procedure. Accordingly, in many cases, if the service provider is not proactively working to offset the increase in the costs of performing procedures, such costs can significantly reduce the provider's profitability.

While many service providers charge and receive the full payment for a procedure, or a fee for service charge (e.g., where a provider's client does not have insurance), in other cases, service providers receive at least a portion of their income from their client's insurance company. In such cases, the amount of money that the service provider receives for a certain procedure may vary significantly from one insurance company to another. Also, while a specific insurance company may be willing to pay enough money for a certain procedure to allow the provider to make a profit on that procedure, for another procedure, the same insurance company may only be willing to pay a fee that will result in a net loss to the provider for performing that procedure. As a result, it may be difficult and time consuming for the provider to keep track of which procedures are profitable to perform and which insurance companies are profitable to accept. As a result, such providers may unintentionally perform a significant number of procedures that result in a net loss to the provider. Similarly, such providers may unintentionally accept an insurance company that results in less profit to the provider than is available from a competing insurance company.

Thus, while techniques currently exist that are used to determine profitability of certain procedures and insurance companies, challenges still exist. Accordingly, it would be an improvement in the art to augment or even replace current techniques with other techniques.

### SUMMARY OF THE INVENTION

The present invention relates to systems and methods for providing financial analyses to service providers. In particular, some implementations of the present invention relate to systems and methods for determining the profitability or loss associated with certain procedures and certain insurance companies.

In some non-limiting instances, implementation of the present invention takes place in association with systems that gather information, for a chosen period of time, about a subset of chosen procedures that are selected from all of the procedures that are performed by a service provider's practice. This gathered information can include, among other things, a time required for the provider to perform each of the chosen procedures, a fee for service charge the provider charges for each chosen procedure; the fees an insurance company is willing to pay for each of the chosen procedures; and a number of each of the chosen procedures the provider performed in the chosen period. In some case, the systems determine a weighted profit/loss for each of the chosen procedures under the fee for service charge and under fee that the insurance company is willing to pay.

While the methods and processes of the present invention have proven to be particularly useful in the area of the healthcare field (including, without limitation, dentistry), those skilled in the art can appreciate that the methods and processes can be used in a variety of different applications and in a variety of different areas where a person, group, or entity provides a good or service. Indeed, the described systems and methods can be used in virtually any field in which a service provider provides a service. Some examples of such providers include lawyers, doctors, therapists, chiropractors, mechanics, servicemen, handymen, plumbers, contractors, construction workers, engineers, and a wide variety of other people and entities that charge money in return for services provided.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a representative system that provides a suitable operating environment for use of the present invention;

FIG. 2 illustrates a representative embodiment of a networked system used in some embodiments of the present invention;

FIG. 3 illustrates a flow chart of a representative method for performing a financial analysis for a service provider;

FIGS. 4-8 each illustrate a representative embodiment of a graph depicting data generated as part of some embodiments of the financial analysis;

FIG. 9 illustrates a representative embodiment of a chart, recommending fee increases; and

FIG. 10 illustrates a representative service plan in accordance with a representative embodiment of the present invention.

### DETAILED DESCRIPTION OF THE INVENTION

The present invention relates to systems and methods for providing financial analyses to service providers. In particular, some embodiments of the present invention relate to systems and methods for determining the profitability or loss associated with certain procedures and certain insurance companies.

In the disclosure and in the claims, the term “service provider” (and variations thereof) may refer to any person, entity, association, group, etc. that provides a service or good in return for a fee. Moreover, the term service provider may, in some cases, also refer to the provider's practice or anyone who is working on the service provider's behalf (e.g., an assistant, nurse, secretary, consultant, analyst, staff, etc.). Some non-limiting examples of service providers include dentists, orthodontists, endodontists, doctors, psychiatrists, nutritionists, social workers, therapists, chiropractors, lawyers, accountants, mechanics, plumbers, contractors, construction workers, handymen, disaster clean up providers, and/or surgeons. In some embodiments, however, the term service provider (and variations thereof) also refers to a person or entity that accepts insurance payments for the provision of services.

The terms service and procedure (and variations thereof) may also be used interchangeably herein to refer to an act, action, transaction, good, material, and/or other thing of value that a service provider provides to, or for, a client or other customer.

As used herein, the term insurance company (and variations thereof) may refer to any entity and/or policy of that entity that makes payments for services on behalf of a recipient of services rendered. Some non-limiting examples of insurance companies include health insurance companies (e.g., BlueCross BlueShield, AETNA, etc.), governmental insurances (e.g., Medicaid, Medicare, Children's Health Insurance Program, etc.), prepaid legal insurance, automobile insurance, and any other known or novel provider of insurance payments.

The following disclosure of the present invention is grouped into two subheadings, namely “Representative Operating Environment” and “Systems and Methods for Performing Financial Analysis.” The utilization of the subheadings is for convenience of the reader only and is not to be construed as limiting in any sense.

### Representative Operating Environment

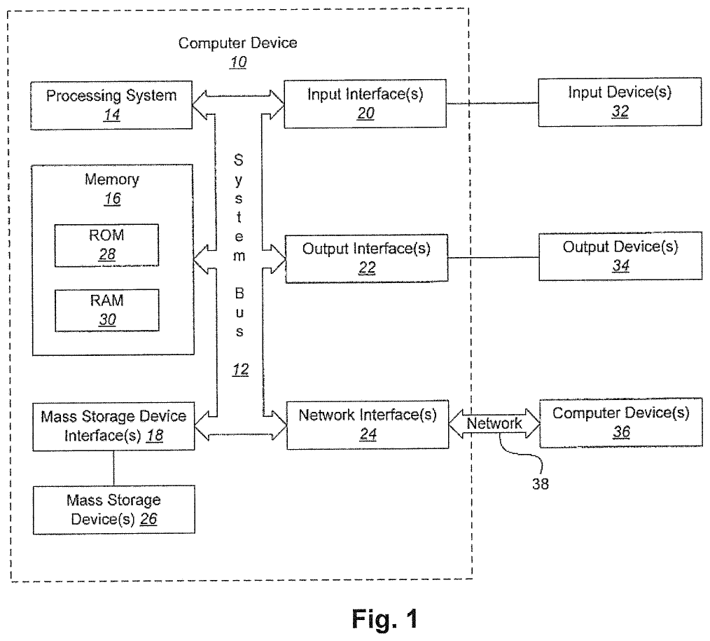

FIG. 1 and the corresponding discussion are intended to provide a general description of a suitable operating environment in which the invention may be implemented. One skilled in the art will appreciate that the invention may be practiced by one or more computing devices and in a variety of system configurations, including in a networked configuration.

Embodiments of the present invention embrace one or more computer readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system.

With reference to FIG. 1, a representative system for implementing the invention includes computer device 10, which may be a general-purpose or special-purpose computer. For example, computer device 10 may be a personal computer, a notebook computer, a personal digital assistant (“PDA”) or other hand-held device, a workstation, a minicomputer, a mainframe, a supercomputer, a multi-processor system, a network computer, a processor-based consumer electronic device, or the like.

Computer device 10 includes system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces 20, output interfaces 22, and/or network interfaces 24, each of which will be discussed below.

Processing system 14 includes one or more processors, such as a central processor and optionally one or more other processors designed to perform a particular function or task. It is typically processing system 14 that executes the instructions provided on computer readable media, such as on memory 16, a magnetic hard disk, a removable magnetic disk, a magnetic cassette, an optical disk, or from a communication connection, which may also be viewed as a computer readable medium.

Memory 16 includes one or more computer readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include hard disk drives, magnetic disk drives, tape drives and optical disk drives. A mass storage device 26 may read from and/or write to a magnetic hard disk, a removable magnetic disk, a magnetic cassette, an optical disk, or another computer readable medium. Mass storage devices 26 and their corresponding computer readable media provide nonvolatile storage of data and/or executable instructions that may include one or more program modules such as an operating system, one or more application programs, other program modules, or program data. Such executable instructions are examples of program code means for implementing steps for methods disclosed herein.

One or more input interfaces 20 may be employed to enable a user to enter data and/or instructions to computer device 10 through one or more corresponding input devices 32. Examples of such input devices include a keyboard and alternate input devices, such as a mouse, trackball, light pen, stylus, or other pointing device, a microphone, a joystick, a game pad, a satellite dish, a scanner, a camcorder, a digital camera, and the like. Similarly, examples of input interfaces 20 that may be used to connect the input devices 32 to the system bus 12 include a serial port, a parallel port, a game port, a universal serial bus (“USB”), a firewire (IEEE 1394), or another interface.

One or more output interfaces 22 may be employed to connect one or more corresponding output devices 34 to system bus 12. Examples of output devices include a monitor or display screen, a speaker, a printer, and the like. A particular output device 34 may be integrated with or peripheral to computer device 10. Examples of output interfaces include a video adapter, an audio adapter, a parallel port, and the like.

One or more network interfaces 24 enable computer device 10 to exchange information with one or more other local or remote computer devices, illustrated as computer devices 36, via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, where functions or tasks are performed by a plurality of networked computer devices.

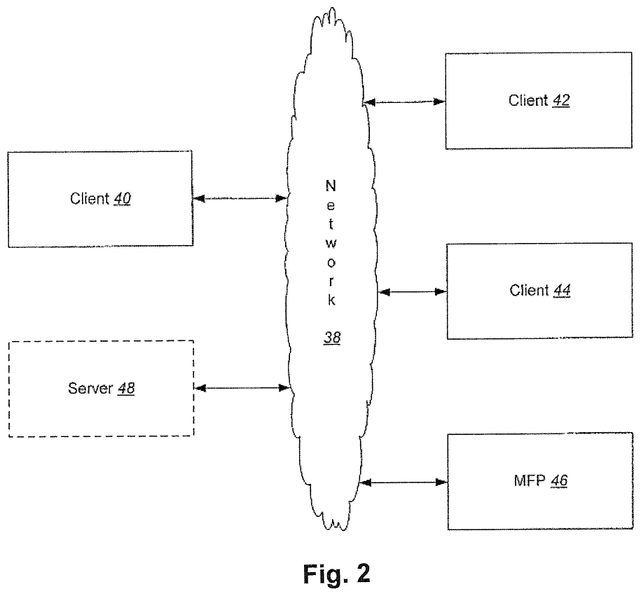

While those skilled in the art will appreciate that the invention may be practiced in networked computing environments with many types of computer system configurations, FIG. 2 represents an embodiment of the present invention in a networked environment that includes clients connected to a server via a network. While FIG. 2 illustrates an embodiment that includes two clients connected to the network, alternative embodiments include one client connected to a network or many clients connected to a network. Moreover, embodiments in accordance with the present invention also include a multitude of clients throughout the world connected to a network, where the network is a wide area network, such as the Internet. Accordingly, in some embodiments, a service provider (or another interested party) is able to use the described systems and methods over a network (e.g., the Internet, the cloud, a web-based service, a platform as a service, a software as a service, etc.) to perform a financial analysis of the provider's practice.

### Systems and Methods for Performing Financial Analysis

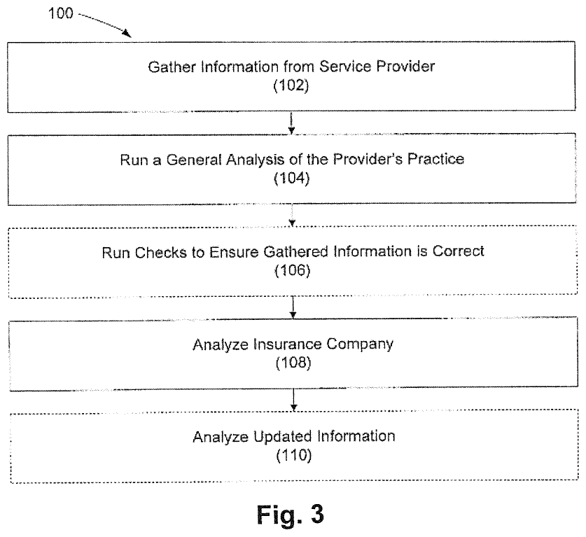

As discussed above, embodiments of the present invention embrace systems and methods for providing financial analyses to service providers. In particular, some embodiments of the present invention relate to systems and methods for determining the profitability or loss associated with certain procedures and certain insurance providers. FIG. 3 shows a representative embodiment of a method 100 for providing a financial analysis for a service provider. Although this method (as well as any portion thereof) can be modified in any suitable manner (including by rearranging, reordering, adding to, removing, modifying, substituting, and otherwise modifying various portions of the method), FIG. 3 shows an embodiment in which the method begins at 102 by receiving information about the service provider.

In this regard, the described systems can gather or receive any suitable information about the provider that will allow the described systems to determine the profitability and/or loss that is associated with certain services and/or with certain insurance companies that the service provider does or does not now accept. Some examples of such information include, but are not limited to: the gross income earned by the provider during a chosen time period (e.g., a year or another suitable amount of time, such as an hour, day, month, quarter, 6-month period, etc.), the actual expenses accrued by the provider during the chosen time period, a list of certain “chosen” procedures that are selected from all of the procedures the provider performs, a total number of each of the chosen procedures that the service provider performs during the chosen time period, the amount of time that it takes the provider to perform each of the chosen procedures, the total time the provider works during the chosen period, the provider's fees for each of the chosen procedures, a list of chosen insurance companies, the gross income the provider has earned through individual insurance companies in the chosen time period, and/or any suitable combination of the foregoing; each of which is discussed below in more detail.

With respect to the provider's gross income (or reported gross income), in some embodiments, the income comprises the gross income the provider earns during the chosen period from all of the procedures the provider performed during that period. More particularly, in some embodiments, the provider's income includes the provider's gross income earned from the provider's provision of services, and excludes any personal cash management income (e.g., non-business related income, such as investment income, loans, loan interest, non-business related depreciation, non-employee retirement plans or 401Ks, non-employee medical premiums or other expenses, and income earned outside of the practice being analyzed). While this reported gross income information can be obtained in any suitable manner, in some embodiments, it is taken (manually or via electronic importation) from the provider's tax information (e.g., 1040 form), financial records (e.g., one or more term sheets and/or profit/loss statement), and/or financial software (e.g., QUICKBOOKS® software). In one non-limiting example, Table 1 shows the provider's gross income was reported to be $10,000 in the chosen period (e.g., one year).

TABLE 1
Chosen Period: Last Year
Reported Gross Income During Chosen Period: $10,000
Reported Gross Income During Chosen Period: $7,000
Reported Hours Worked During Chosen Period: 500
Total Number of Chosen Procedures Performed In Chosen Period: 300

| | | # | Provider's | Total | Fee for |
|---|---|---|---|---|---|
| Code | Procedure Name | Procedures | Time/Procedure(Min) | Time/Procedure | Service Charge |
| 120 | Periodic Oral Eval. | 100 | 25 | 30 | $25 |
| 140 | Limited Oral Eval. | 25 | 50 | 50 | $75 |
| 150 | Comprehensive Eval. | 75 | 120 | 200 | $100 |
| 220 | Intra Oral 1^{st} Film | 100 | 60 | 80 | $50 |

In some embodiments, the actual expenses accrued by the provider include all of the expenses the provider accrued during the chosen time period, less personal cash management expenses (or non-business related expenses). For instance, the provider's actual expenses may include all of the provider's expenses (such as the payroll of all employees, contractors, etc.; worker's compensation fees for employees; rent; utilities; administrative supplies; supplies; tools; taxes; license fees; employee pensions, employee 401Ks, employee bonuses, marketing, etc.; insurance for employees; property insurance; malpractice insurance; bank service costs; other operational costs; or any suitable combination thereof), less the provider's personal income (e.g., the provider's draw); the provider's pension, 401K, etc.; the provider's personal insurances (e.g., the provider's: personal/family insurance premiums, disability insurance, life insurance, etc.); principal payments on any loans; the provider's workman's compensation costs; other expenses that are the provider's personal expenses; or any suitable combination thereof. Thus, in some embodiments, the actual costs include those costs that are actually accrued to run the provider's business and do not include costs that personally benefit the provider. Additionally, in some embodiments, the described systems and methods do not separate fixed and variable costs/expenses. Instead, in such embodiments, the described systems take all costs/expenses during the chosen period and treat them as fixed or averaged costs. Indeed, over a period of time (e.g., the chosen period), supply costs, and other costs, tend to even out. By treating all costs as fixed costs, some embodiments of the described systems can make it easier to gather information from the provider, and can make the calculation process simpler.

While this actual expense information can be obtained in any suitable manner, in some embodiments, it is taken (manually or via electronic importation) from the provider's tax information, financial records, and/or financial software. In one non-limiting example, Table 1 shows the provider's gross expenses were reported to be $7,000 during the chosen period (e.g., one year). Thus, the provider associated with Table 1 had a net profit of $3,000 during the chosen period (e.g., $10,000-$3,000).

Regarding the list of “chosen” procedures, unlike some potential methods that may gather information on each and every procedure the provider performs (or is capable of performing), in some embodiments, the described methods involve collecting and analyzing a limited list of procedures. Where the list includes a relatively small number of procedures and/or excludes some procedures performed by the provider, it may be relatively easy to gather information about the chosen procedures.

The list of chosen procedures can include any desired procedures, including, without limitation, the most-frequent procedures provided by the provider, the provider's favorite procedures, procedures with a high production value, a random sampling of the procedures provided by the provider, etc. Moreover, the list of chosen procedures can include any suitable number of procedures. Indeed, while in some embodiments, the list includes between about 1 and about 1,000 procedures, in other embodiments, the list includes less than about 100 procedures, or even less than about 50 procedures (e.g., about 25±15). In still other embodiments, the list of chosen procedures includes any suitable combination or sub-combination of the aforementioned ranges. By way of non-limiting illustration, Table 1 shows an example in which the list of chosen procedures contains 4 procedures, namely a periodic oral evaluation, a limited oral evaluation, a comprehensive evaluation, and an intra-oral 1^{st} film.

Regarding the total number of each of the chosen procedures the service provider performs during the chosen time period, in some instances, this number is simply the total number (or an estimate) of the number of each of the chosen procedures performed in the chosen period. By way of non-limiting illustration, Table 1 shows an example in which the provider performed 100 periodic oral evaluations, 25 limited oral evaluations, 75 comprehensive evaluations, and 100 intra-oral 1^{st} films.

With respect to the amount of time that it takes the provider to perform each of the chosen procedures, in some embodiments, the provider gives an estimated time that it takes the provider to complete each procedure. In other embodiments, however, the provider indicates how much time it actually bills on the procedure—whether or not it actually takes the provider more or less time to perform the procedure. Additionally, in some embodiments, in order to keep from duplicating time spent on each of the chosen procedures by the provider and the provider's employee, staff, assistants, etc. (or even another provider), the described systems and methods seek to obtain the time the provider spends on a procedure (and not the time spent by the employee's, staff, assistants, etc. (or another provider). For instance, if a dental hygienist typically spends 5 minutes of the 30 minutes of total time allotted to a procedure (e.g., a periodic oral exam, as shown in Table 1), and the dentist performs the other 25 minutes of the procedure (as also shown in Table 1), then some embodiments of the described systems would ignore the 5 minutes spent by the hygienist and instead would use the 25 minutes spent by the dentist in the calculations.

With regards to the total time the provider works during the chosen period, in some embodiments, this number includes all of the time the provider is at work. Indeed, in some embodiments, the total time the provider works during the chosen period includes the time spent in which the provider is performing procedures (e.g., dental procedures), meeting with clients, performing administrative tasks (e.g., bills, trainings, etc.), and simply passing time (e.g., waiting for the next client). In one non-limiting example, Table 1 shows the provider worked 500 hours during the chosen period (e.g., the last year).

With regard to the fees for each of the chosen procedures, in some embodiments, the provider gives a list of fixed fees or fee for service charges (as opposed to a range or a list of variable costs) for each of the chosen procedures. By way of illustration, Table 1 (shown above) shows a non-limiting example of a list of fees (e.g., fee for service charges) in which the provider charges $25 for a periodic oral evaluation, $75 for a limited oral evaluation, $100 for a comprehensive evaluation, and $50 for intra-oral 1^{st} films.

Continuing with step 102 in FIG. 3, in some embodiments in which the provider accepts insurance payments (e.g., dental insurance, medical insurance, etc.), the provider also provides a list of chosen insurance companies. Although in some instances, this list only includes the insurance companies the provider currently accepts, in other instances, the list includes insurance companies that the provider does not currently accept (e.g., insurances that the provider may potentially accept). In some embodiments, the provider optionally indicates (for the chosen time period) the gross income the provider earned through each of the individual insurance companies that the provider accepted during that period of time.

As the method 100 progresses, step 104 of FIG. 3 shows that in some embodiments, the described methods include running a general analysis of the provider's practice. While this step can include any suitable calculation, in some embodiments, it includes calculating the provider's gross income per hour (i.e., by dividing the provider's reported gross income in the chosen period by the total number of hours worked in the period (e.g., $10,000/500=$20/hour, for the provider of Table 1)); the provider's expense per hour (i.e., by dividing the gross expense for the chosen period by the total hours worked in that period (e.g., $7,000/500=$14/hour, for the provider of Table 1)); the provider's net profit for each hour in the chosen period (i.e., by subtracting the expense per hour in the chosen period from the income per hour in that period, (e.g., $20/hour-$14/hour=$6/hour net profit)); the average expense per procedure (i.e., by dividing the gross expense for the chosen period by the total number of chosen procedures performed in that period (e.g., $7,000/300=$23.33/procedure, for the provider of Table 1)); the provider's gross income per chosen procedure during the chosen period (i.e., by dividing the gross income of the chosen period by the total number of procedures performed during that period (e.g., $10,000/300=$33.33/procedure)); the provider's percent expense (i.e., by dividing the provider's gross expense of the chosen period by the provider's gross income of that period (e.g., for the provider of Table 1, $7,000/$10,000=70%)); and/or the provider's overall net profit percent during the chosen period (i.e., by dividing the provider's net profit during the chosen period by the provider's gross income during that period (e.g., $3,000/$10,000=30%).

Step 106 in FIG. 3 shows that, in some embodiments, the method 100 optionally includes checking the information provided above in step 102 to ensure that such information is correct. While this process can check any suitable portion of the information, in some non-limiting embodiments, this process checks to see that the reported time to perform the chosen procedures is correct and that the reported gross income for the chosen period is correct.

While the reported time to perform the chosen procedures can be checked in any suitable manner, in some embodiments, this process includes determining the number of hours the provider would have worked if the reported time for each procedure were correct when used with the actual number chosen procedures the provider provided during the chosen period. By way of non-limiting example, according to the procedure times and numbers of each procedure that are set forth in Table 1, during the chosen period, the provider would have spent: about 42 hours performing period oral evaluations (e.g., (25 minutes/60 minutes per hour)×(100 oral evaluations)=42 hours of oral evaluations); about 20.75 hours performing limited oral evaluations (e.g., (50 minutes/60 minutes per hour)×(25 limited oral evaluations)=20.75 hours spent performing limited oral evaluations); about 150 hours performing comprehensive evaluations (e.g., (120 minutes/60 minutes per hour)×(75 comprehensive evaluations)=150 hours spent performing comprehensive evaluations); and about 100 hours performing intra-oral 1^{st} films (e.g., (60 minutes/60 minutes per hour)×(100 intra-oral 1^{st} films)=100 hours performing intra-oral 1^{st} films. Thus, in this example, while the provider reportedly worked 500 hours in the chosen period, the reported time per procedure and reported number of procedures indicates that the provider only performed the chosen procedures for 312.75 hours (e.g., 42+20.75+150+100=312.75 hours that were calculated to have been spent on the chosen procedures).

In some embodiments, where there is a discrepancy between the reported hours worked (e.g., 500 hours) and the calculated time spent on chosen procedures (e.g., 312.75 hours), a freedom time multiplier is used to adjust or correct the reported time spent on each procedure. While the freedom time multiplier can be determined in any suitable manner, in some embodiments, it is determined by dividing the reported number of hours worked by the calculated time spent on chosen procedures. By way of non-limiting example, where the provider of Table 1 is reported to have worked 500 hours and the calculated time spent on the chosen procedures was 312.75 hours, the freedom time multiplier is about 1.6 (e.g., 500/312.75). Once the freedom time multiplier has been calculated, the reported time for each procedure can be corrected to create an adjusted time per procedure (as shown in Table 2) by being multiplied by that multiplier (e.g., for periodic oral evaluations (25 minutes per evaluation/60 minutes per hour)×1.6=0.67 hours (as shown below in Table 2)).

TABLE 2

| | | | | | Adiusted | Adiusted | | % of | |
|---|---|---|---|---|---|---|---|---|---|
| | | Reported | Reported | Freedom | Time/ | Expense | Hours/procedure/ | Total or | Hours/procedure/ |
| | | Time | Time | Time | Procedure | per | Yr (from Adjusted | Weight | Yr (From Weight |
| Code | Name | (min) | (Hr) | Multiplier | (Hr) | Procedure | Time/Procedure) | % | % of each) |
| 120 | Periodic Oral Eval. | 25 | 0.42 | 1.6 | 0.67 | $9.30 | 67 | 33.33% | 166.67 |
| 140 | Limited Oral Eval. | 50 | 0.83 | 1.6 | 1.33 | $18.70 | 33.25 | 8.33% | 41.67 |
| 150 | Comprehensive Eval. | 120 | 2.0 | 1.6 | 3.2 | $44.8 | 240 | 25% | 125 |
| 220 | Intra-Oral 1^{st} Film | 60 | 1.0 | 1.6 | 1.6 | $22.40 | 160 | 33% | 166.67 |
| | | | | | | | 500 | 100% | 500 |

With the adjusted time per procedure (or the reported time per procedure, if already correct), the described systems and methods can calculate a number of useful calculations. In this regard, Table 2 shows that such calculations may include, but are not limited to, the un-weighted cost per procedure (i.e., expense per hour multiplied by the adjusted time per procedure, (e.g., $14×0.67=$9.30 for the periodic oral evaluations of Tables 1 and 2)); hours per procedure per chosen time period, from the total time (i.e., the adjusted time per procedure multiplied by the total number of each procedure (e.g., 0.67×100=67, for periodic oral evaluations of Tables 1 and 2)); the percent of the total procedures for each chosen procedure, or a total weighted percent (i.e., by dividing the number of each chosen procedure performed in a chosen period by the total number of chosen procedures performed in that period (e.g., for periodic oral evaluations in Table 1, 100/300=33.33%)); and/or the hours per procedure during the chosen period (from the weighted percent of each) (i.e., by multiplying the total number of hours worked in the chosen period by the weighted percent of the individual chosen procedures (e.g., 500 hours×33.33%=166.65 for the periodic oral evaluations of Tables 1 and 2)).

Where the described systems and methods check the reported income, the reported income can be verified in any suitable manner. In some embodiments, this check involves calculating the “factored volume” of each of the chosen procedures. While this can be done in any suitable manner, in some embodiments, it involves taking the number of a particular chosen procedure that the provider is reported to have performed in the chosen time period and then multiplying that number by the amount of time that it takes the provider to perform that procedure (e.g., the adjusted time per procedure). For instance, if a dentist performs 100 periodic oral evaluations each year, and each evaluation takes 0.67 hours, then the dentist's factored volume for extractions is 67 (e.g., 100×0.67=67). Such a process can be repeated for each of the chosen procedures.

In some embodiments, the process for checking the reported income continues by calculating the adjusted expense for each of the chosen procedures. Although this number can be calculated in any suitable manner, in some instances, it is calculated for each of the procedures in two steps. In some cases, the first step includes dividing (individually) the factored volume of each of the chosen procedures by the sum of the factored volumes for all of the chosen procedures (e.g., 67/500=0.134). Additionally, in some embodiments, the second step involves taking the total from the first step (e.g., 0.134) and multiplying it by the reported gross reported expense for the chosen period (e.g., $7,000) divided by the total number of each of the corresponding chosen procedures that were performed in the chosen period (e.g., $7,000/100=$70, where 100 is the number of reported number of periodic evaluations, thus 0.134×$70=$9.33 (as shown in Table 2)). In some instances, this step is repeated for using the numbers corresponding to each of the chosen procedures.

In some cases, the process for checking the reported income continues by calculating the total expense for each chosen procedure during the chosen time period” (e.g., one year). In some embodiments, this step is accomplished by individually multiplying the reported number of each procedure by the adjusted expense for that procedure. By way of non-limiting example, where the provider performs 100 periodic oral evaluations during the chosen period, and the and the adjusted expense for each evaluation is $9.33, then the total expense for each periodic evaluation in the chosen period (e.g., one year) is 100×$9.33=$933 (as shown below in Table 3).

In some embodiments, the process for checking the reported income continues by calculating the revenue earned during the chosen period (e.g., the last year) for each of the chosen procedures. While these revenues can be calculated in any suitable manner, in some embodiments, they are calculated by multiplying the provider's set fee (e.g., fee for service charge) for a chosen procedure by the corresponding number of that procedure that the provider performs during the period, and then repeating this for each of the chosen procedures. For example, where the provider charges $25 (as shown below in Table 3) for each periodic evaluation and performs 100 periodic evaluations during the chosen period (e.g., one year), the provider's revenue for such evaluations during the chosen period is $2,500 (e.g., $25×100).

In some embodiments, the described systems and methods further calculate the provider's total possible income (based on fee for service charges) during the chosen period by adding up the provider's income for each of the chosen procedures during the chosen period (e.g., as shown below in Table 3, $2,500+$1,875+$7,500+$5,000=$16,875). This calculated gross income in the chosen period is the maximum income the provider could have earned in the chosen period (given the reported information). Additionally, this calculated total possible income can then be compared with the provider's reported income to ensure that the provider actually reported the right income. By way of non-limiting example, where the gross income of the provider is reported as being $10,000 and the calculated gross income is determined to be $16,875, the system can detect the discrepancy.

TABLE 3

| | | | Provider's Fee | Adjusted | | | | Net |
|---|---|---|---|---|---|---|---|---|
| | | # | for Service | Expense/ | | | Adiusted | Profit/Yr/ |
| Code | Name | Procedures | Charges | Procedure | Yearly Expense | Income/Yr | Net Profit | Procedure |
| 120 | Periodic Oral Eval. | 100 | $25 | $9.30 | $933.30 | $2,500 | $15.70 | $1,566.70 |
| 140 | Limited Oral Eval. | 25 | $75 | $18.70 | $446.70 | $1,875 | $56.30 | $1,408.30 |
| 150 | Comprehensive Eval. | 75 | $100 | $44.80 | $3,360.00 | $7,500 | $55.20 | $4,140.00 |
| 220 | Intra-Oral 1^{st} Film | 100 | $50 | $22.40 | $2,240.00 | $5,000 | $27.60 | $2,760.00 |
| Totals: | | 300 | | | $7,000 | $16,875 | $154.80 | $9,875.00 |

In some cases, when the described systems and methods detect a discrepancy (or at least a significant discrepancy) between the calculated gross income and the reported gross income, some embodiments of the described systems flag the discrepancy and/or seek to identify the cause of the discrepancy. In some embodiments, as some insurance companies pay less than the provider's set fee (fee for service charge), and hence prevent the provider from ever realizing the calculated possible gross income, the described systems and methods check to see if the reported income is higher than the calculated gross income. By way of non-limiting example, where the provider's reported gross income is greater than the calculated gross income, some embodiments of the described systems and methods try to identify income/expenses that were reported and are not related to the provider's actual practice (e.g., that are cash management numbers).

In some embodiments, the process for checking the gathered information further includes calculating the adjusted net un-weighted profit/loss for each of the chosen procedures. While this can be accomplished in any suitable manner, in some instances, this is accomplished by subtracting the adjusted expense for a chosen procedure from the provider's set fee (or fee for service charge) for that procedure, and then repeating that process for each of the chosen procedures. By way of non-limiting example, Table 3 (above) shows that where the provider charges $25 for a periodic evaluation and the adjusted expense for each evaluation is $9.38, then the provider's adjusted net profit per periodic evaluation is $15.62 (e.g., $25−$9.38=$15.62).

In some embodiments, the process for checking the initial information continues by calculating the net profit generated by each of the chosen procedures during the chosen period (e.g., one year). In some instances, the net profit for each of the chosen procedures is determined by multiplying the adjusted net profit for one of the chosen procedures by the number of times that the particular procedure is performed in the period. In some embodiments, this process is also repeated for each of the chosen procedures. By way of non-limiting example, Table 3 (above) shows that where the adjusted net profit for periodic evaluations is $15.62 and the number of such evaluations performed in the chosen period (e.g., one year) is 10, the provider's net profit for such evaluations during the chosen period is $1,562 (e.g., $15.62×100).

In some embodiments, the net profits per chosen period for each of the chosen procedures are added together to calculate the provider's calculated net profit for the chosen period (e.g., as shown above in Table 3, $1,562.00+$1408.30+$4,140.00+$2,760.00=$9,870.30).

Continuing with the method set forth in FIG. 3, step 108 shows that, in some embodiments, the described systems and methods conduct an evaluation of any insurance company that the provider currently accepts or may possibly accept. While this can be done in any suitable manner, in some embodiments, this process of evaluating the various insurance companies involves calculating a weighted profit/loss for each of the chosen procedures. In some instances, this is accomplished by multiplying the net profit/loss per procedure by the corresponding weight percent of each procedure. By way of non-limiting example, where the net profit/loss for a procedure (e.g., the periodic oral evaluation) is $15.62 and the weight % of that procedure is 33.33%, then the weighted profit/loss for that procedure (as shown below in Table 4) is about $5.21 (e.g., $15.62×33.33%). In some embodiments, such a process is repeated for each of the chosen procedures. Moreover, in some embodiments, such a process is repeated for each of the chosen procedures during the chosen period, for each of the chosen insurance companies—using (as a basis for calculating the weighted profit/loss of the procedures) the fee the insurance companies are willing to pay for each of the chosen procedures.

TABLE 4

| | | | Profit/Loss per | Profit/Loss per | Profit/Loss per |
|---|---|---|---|---|---|
| | | Fee for Service | Procedure for Fee | Procedure for | Procedure for |
| Code | Name | Charge | for Service | Insurance A | Insurance B |
| 120 | Periodic Oral Eval. | $25 | $5.22 | $4.89 | $5.22 |
| 140 | Limited Oral Eval. | $75 | $4.69 | $0.94 | $1.94 |
| 150 | Comprehensive Eval. | $100 | $13.80 | −$2.70 | −$0.20 |
| 220 | Intra-Oral 1^{st} Film | $50 | $9.20 | −$4.13 | −$3.13 |

| | | | |
|---|---|---|---|
| Weighted Net Profit Margin | 58.52% | −1.78% | 6.81% |
| For Every $100 Charged Provider Takes Home: | $58.52 | −$1.78 | $6.81 |
| Sum of All Fees Allowed (Weighted) | $32.92 | −$1.00 | $3.83 |

In some embodiments, as the described systems compare the provider's fees for service revenues against the revenues that are earned or available through insurance companies, the described systems obtain a weighted total for each procedure. While this weighted total can be calculated in any suitable manner, in some embodiments, it is calculated for the provider's fee for service charges by multiplying weight percent of a certain procedure by the provider's corresponding fee for service charge for that service. For instance, where the provider's fee for service for a procedure (e.g., a periodic evaluation) is $25.00 and the weighted percent for that procedure is 33.33% (as shown below in Table 5), then the weighted net profit for that procedure (as shown in Table 5) is about $8.33. In some embodiments, the weighted total for each of the chosen procedures is then added to provide a weighted total for the fee for service charges.

TABLE 5

| | Number of | Weighted | Weighted | Weighted | Profit/Loss |
|---|---|---|---|---|---|
| Code | Procedures | % | Total | Net Profit | Per Procedure |
| 120 | 100 | 33.3% | $8.33 | $5.22 | $15.67 |
| 140 | 25 | 8.3% | $6.25 | $4.69 | $56.33 |
| 150 | 75 | 25.0% | $25.00 | $13.80 | $55.20 |
| 220 | 100 | 33.3% | $16.67 | $9.20 | $27.60 |
| Total: | 300 | 100% | $56.25 | $32.92 | $154.80 |

In some embodiments, the described systems and methods also calculate the weighted total for each of the chosen procedures, when such procedures are paid for by an insurance company. In some embodiments, the weighted total is calculated for each insurance company by multiplying weight percent of a certain procedure by the insurance company's corresponding payment for that service.

In some embodiments, after the described systems and methods have calculated the weighted total and the weighted net profit (or net profit percent) for each of the chosen procedures under the corresponding fees for service charges and the payments of each of the chosen insurance companies, the described systems calculate a weighted net profit margin for the fees for service charges and each of the desired insurance companies. Although this can be accomplished in any suitable manner, in some embodiments, it is accomplished by dividing the sum of the weighted net profit of all of the chosen procedures by the sum of the weighted total for each of the procedures. By way of non-limiting example (and as shown above in Table 5), where the sum of the weighted net profit for each of the chosen procedures (as calculated for the fees for service charges) is $32.92 and the sum of the weighed totals for each of the chosen procedures (as also calculated for the fees for service charges) is $56.25, then the net profit margin after expenses is 58.52% (e.g., $32.92/$56.25), meaning that for every $100 the provider charges under the providers' fee for service charges, the provider takes home about $58.52. In some embodiments, similar calculations are conducted for each of the insurance companies (using the fees such companies will pay for each of the procedures). By way of non-limiting example, Table 4 (above) shows some embodiments, in which the weighted net profit margin for two insurance companies (e.g., Insurances A and B) is determined to be −$1.78 and $6.81, meaning that for every hundred dollars the provider would have charged under the provider's fee for service fee schedule, the provider will lose $1.78 through Insurance A, and will only take home $6.81 through Insurance B. Thus, in some embodiments, the provider can look at the weighted net profit margin for any suitable number of insurances and quickly compare their profitability. While this ability may be useful for several reasons, in some cases, where the provider is busy (e.g., has relatively little open chair time), the provider can determine which (and potentially drop) insurance companies are less profitable.

In some embodiments, the described systems and methods also compare the provider's expense per hour with the income the provider earns from each insurance company per hour. In some such embodiments, the systems calculate the gross income per hour per procedure and the gross income per hour per procedure for each of the insurance companies. With respect to the gross income per hour per procedure under the provider's fee for service charges, in some embodiments, such a number is calculated by dividing the provider's fee for service charge for a particular chosen procedure by the time the provider spends performing the procedure (e.g., the adjusted procedure time). By way of non-limiting example, where the provider charges $25 for a periodic oral evaluation and it takes the provider 0.67 hours to complete that procedure (as shown above in Table 2), the gross income per hour for that procedure (as shown below in Table 6) is $37.50 (e.g., $25/0.67). A similar process can also be completed for each of the chosen procedures. Furthermore, in some embodiments, the gross incomes for each of the chosen procedures can be added up (e.g., $37.50+$56.50+$31.30+$31.30=$156.3).

TABLE 6

| | | | Gross Income/ | | Fee Insurance A | |
|---|---|---|---|---|---|---|
| | | Provider's Fees | Hour/Procedure | | is Willing to | Gross Income/ |
| | | for Service | for Fee for | Insurance A's | Pay for | Hour/Procedure |
| Code | Name | Charges | Service | Fee Schedule | Procedure | for Insurance A |
| 120 | Periodic Eval. | $25.00 | $37.50 | $24.00 | $24.00 | $36.00 |
| 140 | Limited Eval. | $75.00 | $56.30 | $30.00 | $30.00 | $23.00 |
| 150 | Comprehensive Eval. | $100.00 | $31.30 | $34.00 | $34.00 | $11.00 |
| 220 | Intra-Oral 1^{st} Film | $50.00 | $31.30 | $60.00 | $31.30 | $6.00 |

| | | |
|---|---|---|
| Totals | $156.30 | $75.4 |
| Overall Income per Hour | $20 | $10 |
| Expense per Hour | $14 | $14 |

With regards to the gross income per hour per procedure earned through the insurance companies, this calculation can be accomplished in any suitable manner. In some embodiments, however, it is done by dividing the fee a desired insurance company is willing to pay for a procedure by the total time (e.g., adjusted time) that it takes the provider to perform that procedure. In this regard, because the insurance company will typically only pay the lesser of the insurance company's scheduled fee for a procedure and the provider's fee for service charge for that procedure (as shown in for procedure 220 in Table 6), in some embodiments, the described systems and methods also use the lesser of the two (or the fee the insurance company is willing to pay for a procedure). In one non-limiting example of a method for calculating the gross income per hour per procedure earned through an insurance company, where a provider charges a $25 fee for service charge for a procedure (e.g., a periodic oral evaluation), but the insurance company is only willing to pay $24 dollars for the procedure (as shown above in Table 6), then the gross income per hour for that procedure is about $36 (e.g., $24/0.67). Thus, in some embodiments, the provider can quickly look at and compare the gross income for each of the chosen providers earned through fee for service charges, and various insurance companies.

In some embodiments, the described systems and methods also provide a comparison between the overall income per hour earned at the provider's practice and the gross income per hour when the chosen insurance companies pay for a procedure. In this regard, as discussed above, the gross income per hour earned at the practice can be determined by dividing the provider's gross income by the total number of hours worked (e.g., $10,000/500=$20 per hour).

With regards to the overall income per hour for an insurance company this can be calculated in any suitable manner. In some embodiments, it is calculated by dividing the total gross income per hour for each of the chosen procedure for a chosen insurance company by the total gross income per hour for each of the chosen procedures under the fee for service charges, and then multiplying that result of that equation by the provider's gross income per hour (e.g., ($75.4/$156.30)×$20=$10). Thus, the described systems can quickly allow a provider to compare the overall income per hour of the practice and each of the chosen insurance companies. For instance, where the provider's expense per hour is $14, and the overall income per hour of the practice is $20, the provider is making $6 profit each hour. However, where the provider's expense per hour is $14, and the overall income per hour provided by Insurance A is $10, the provider is losing $4 per hour by accepting that insurance. Accordingly, in some embodiments, the described systems allow the provider to compare the overall income per hour of one or more insurance companies against the provider's hourly expenses to see what the hourly loss/gain is with each insurance company. Thus, in some embodiments, the provider is able to see, with a single number, how much money he is earning or losing per hour with each insurance company. While such a calculation may be useful for a variety of reasons, in some instances, this calculation is especially useful where the provider has excessive amounts of down time during the chosen period. For instance, if the provider is able to see that the provider is still paying the hourly expense (e.g., $14/hour) when the provider is not performing procedures (e.g., during open chair time), the provider may be able to see that it is better to offset such costs by accepting another insurance (which in turn will allow the provider to assist more customers)—even if that insurance company is not particularly profitable on a per hour basis when compared to other insurance companies.

Continuing with FIG. 3, step 110 shows that, in at least some embodiments, the described systems and methods optionally allow the provider (or another party) to update any of the gathered information at any time. By way of non-limiting example, in some instances, the provider can change any of the provider's fee for service charges and the described systems could automatically update the analysis to show the provider how such changes would affect the provider's practice. For instance, the provider could check to see what effect raising any specific chosen procedure by any specific amount would have on the practice's overall profitability.

In addition to the aforementioned features, the described systems and methods can be modified in any suitable manner. In one non-limiting example, the described systems and methods can be used to perform any other suitable calculation, including, without limitation, the total income the provider earns from a chosen insurance company during a chosen period (e.g., by multiplying the total number of each of the chosen procedures performed in the chosen period by the corresponding fees the insurance company is willing to pay for each procedure, and then summing up the results for each procedure); and any other calculation that may be interesting or otherwise help a provider to optimize the profitability of the provider's practice

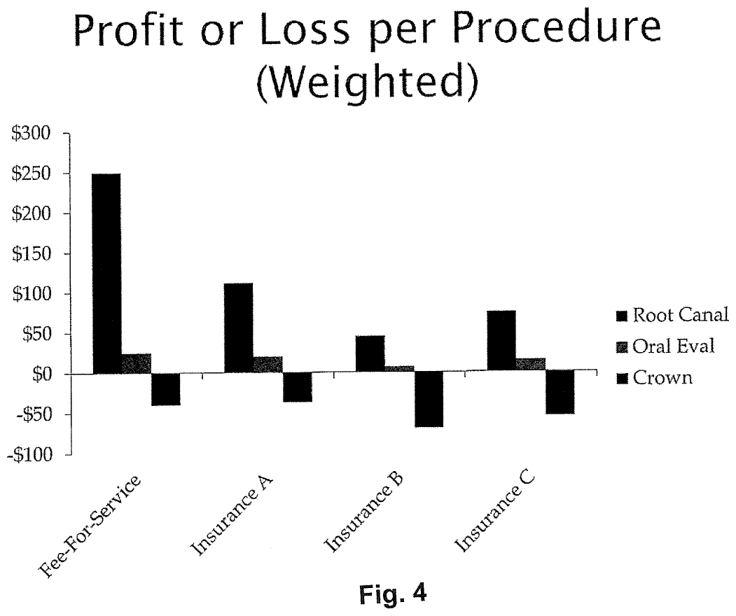

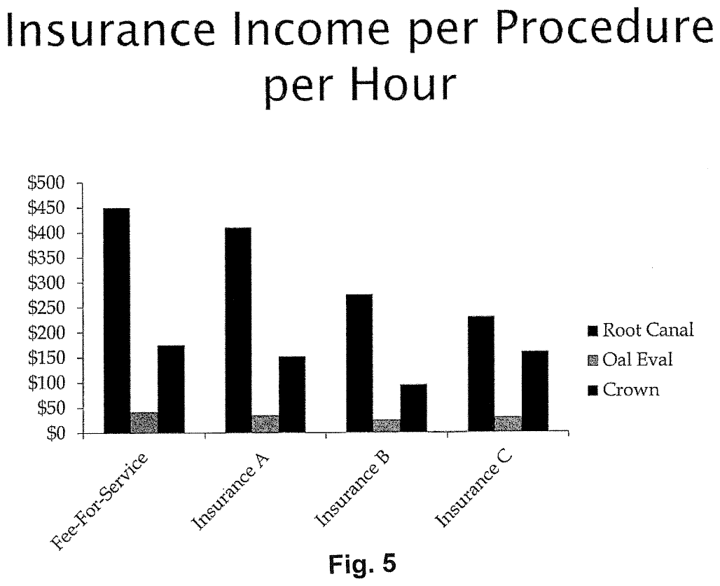

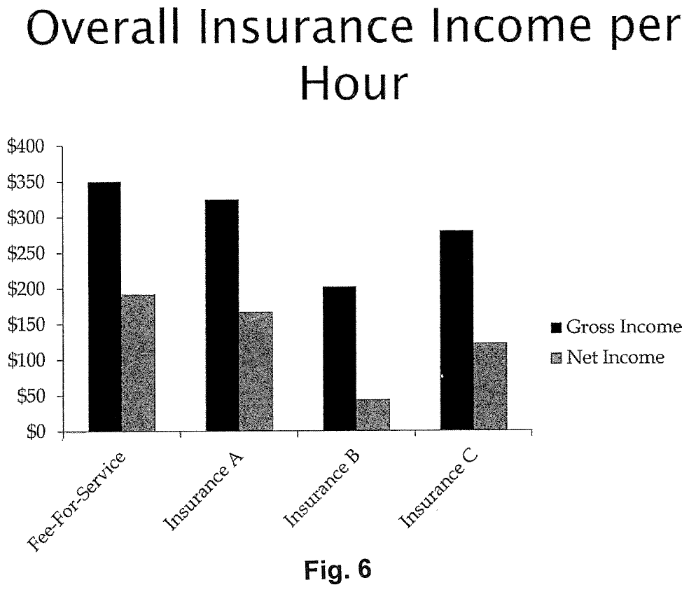

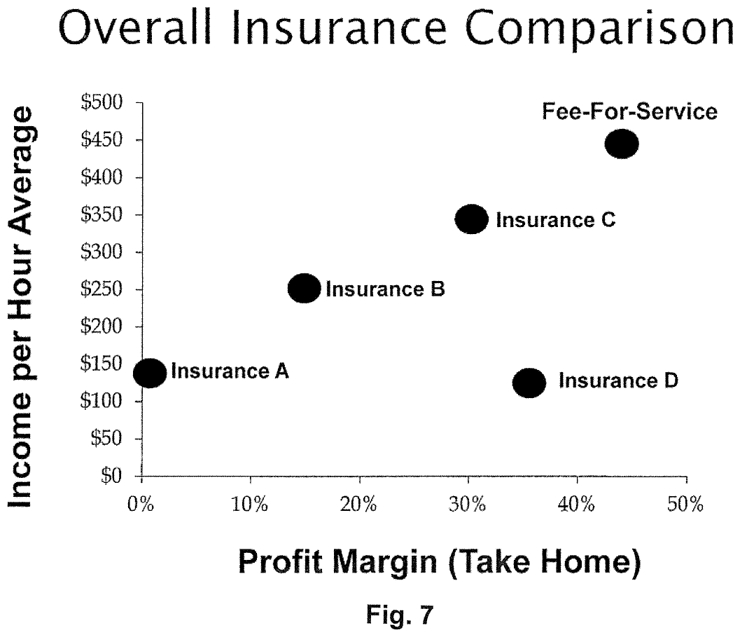

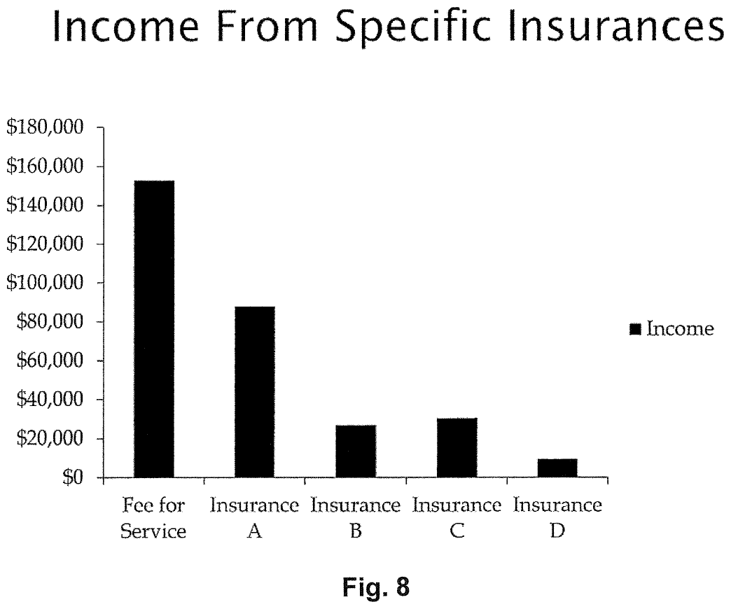

In another non-limiting example of a manner in which the described systems and methods can be modified, in some embodiments they generate graphical representations of some or all of the data that is calculated as part of the described analysis. By way of non-limiting illustration, FIG. 4 shows a representative graph depicting the weighted profit/loss that a provider may earn when providing a few services (e.g., root canals, oral evaluations, and crowns) under fee for service charges and through a few insurance companies. Similarly, FIGS. 5, 6, 7, and 8 respectively depict a graph showing a representative graph depicting: insurance income per hour, per procedure; overall insurance income per hour; an overall insurance comparison; and income from specific insurances.

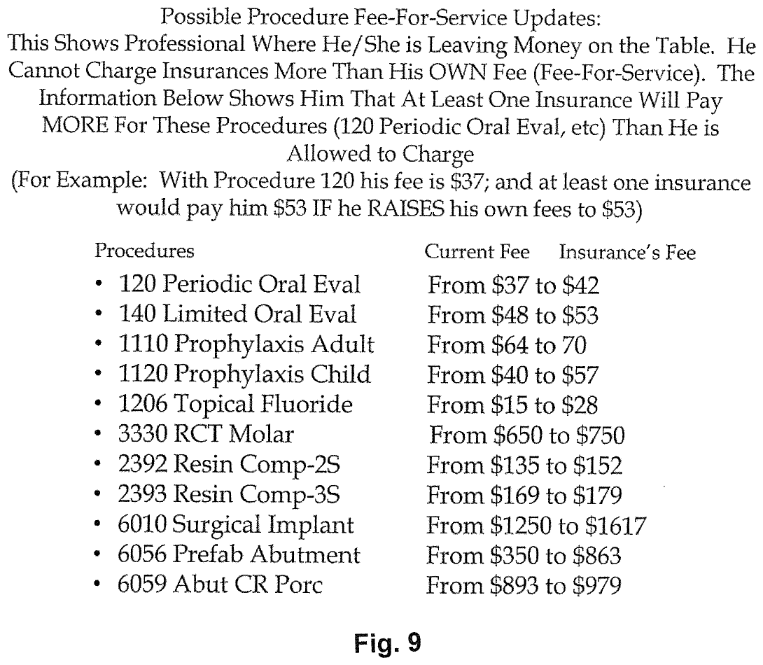

In still another non-limiting example, some embodiments of the describe systems and methods further provide the provider with one or more suggestions that can improve the provider's profitability. In this regard, the described systems can provide any suitable suggestions that allow the systems to function as intended. For instance, FIG. 9 shows that, in some embodiments, if the provider's fee for service charge for a procedure is less than the fee of any of the insurance companies the provider accepts, then the described systems may suggest that the provider raise the provider's fees to match what the insurance companies are willing to pay. Similarly, the described systems may suggest that a provider stop performing certain procedures or using insurance companies that result in a loss to the provider. In contrast, in some embodiments the described systems suggest that the provider focus on certain profitable procedures, referring them out to specialists, and/or pick up one or more insurance companies that would be profitable to the provider. In still other instances, some embodiments of the described systems suggest changes that the provider may make to reach a certain net profit during a future period of time.

In still another non-limiting example, some embodiments of the described systems and methods obtain market updates and provide those updates and/or any related suggestions to the provider.

In at least some embodiments, service providers find it useful to know whether it is profitable to contract with a new insurance company. Such an analysis considers the following: (i) current customers/patients carrying insurance from the new insurance company who are currently considered “out-of-network” because the service provider is not listed as a provider of the new insurance company; and (ii) customers/patients whose insurance is “out-of-network” are paying a service provider's or professional's own fees (referred to as fee-for-service), which is typically higher than the contracted fees associated with the new insurance company.

The following determines the number of new insurance patients the service provider/professional will need to add in order to make up the difference in the income per hour for the fee-for-service they are currently receiving and the insurance's own income per hour.

TABLE 7
The number of current patients carrying the new insurance:
10
Multiply the income per hour (fee-for-service) by the number of current
patients:
$200 × 10 = $2000
Multiply income per hour (new insurance) by the number of Current
Patients:
$100 × 10 = $1000
Determine the difference:
$2000 − $1000 = $1000
Divide the difference by the insurance's income per hour:
$1000/10 = 100

As a result, the service provider/professional would need to add an additional 100 patients at the new insurance's contracted income per hour for it to be profitable for the service provider to contract with the new insurance company.

In some embodiments, a service provider/profession is able to provide their own office service plan.” Some patients are not covered by an insurance plan. Thus, such patients are able to sign up for a promotional rate on a specified set of procedures as well as a discount from the service provider/professionals own fee-for-service for all other services. Unlike many insurance plans, the service professional's service plan would not have a maximum yearly allowance.

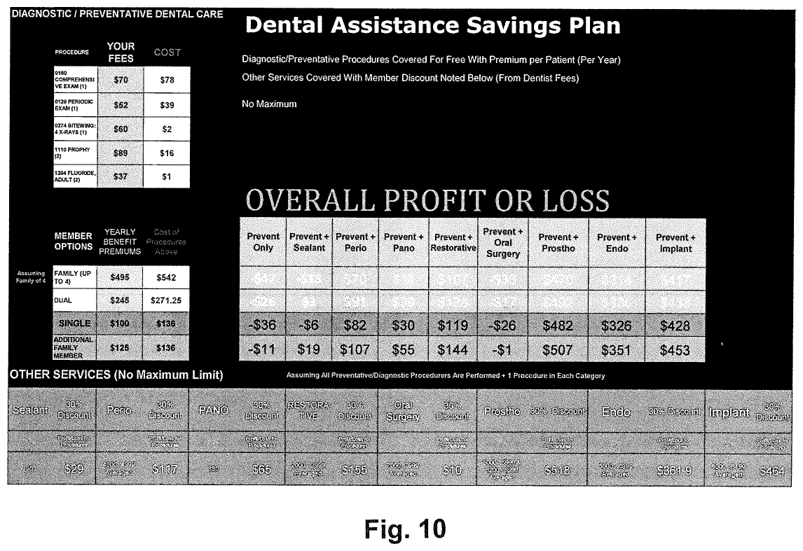

FIG. 10 uses the calculations from the original analysis to total the costs for the procedures in question. By way of example, the “Diagnostic/Preventative Care” refers to five representative procedures in the first set of boxes (upper left). (Those skilled in the art will appreciate that embodiments of the present invention embrace more than five or less than five procedures.) From left to right there exists: (i) the procedures; (ii) the service provider's or professional's fee-for-service fees; and (iii) the cost for each procedure (the cost may be doubled for procedures that will be provided twice in one year).

The costs are summed to give the total cost for the package of procedures. The total cost is then used to set a yearly “promotional” fee for those services.

With reference to the boxes to the bottom left, those under “Member Options” specify the groups to whom the service plan may be offered. For example, a family of four, “Dual” (husband and wife), “Single” (individual), and “Additional Family Member” (each additional person added to the family of four, dual or single).

The next box to the right is where the “promotional rate” (titled “Yearly Premium Benefits”) is set. The next box over “Cost of Procedures Above” is the summed total cost for the five procedures in the first set of boxes. The first row shows the cost multiplied by four, since in the present example it is for a family of four.

In the portion of FIG. 10 that is labeled “Overall Profit or Loss,” the box “Prevent Only” is calculated by subtracting the “Cost of Procedures” from the “Yearly Premium.” This identifies the Profit/Loss if each group (family of four, dual or single) uses all of the diagnostic/preventative procedures during the year. This allows the service provider/professional to set a promotional rate high enough to cover the costs of the service provider with the exact profit/loss that they determine.

Going from right to left across the different categories (Prevent+Sealant, Prevent+Perio, etc), we determine the Profit/Loss for each group (family of four, dual or single) by considering the cost for the Diagnostic/Preventative package of procedures and the Profit or Loss resulting from the patient using one of each procedure in that category (Sealant or Perio, or Pano, etc).

Each grouping's (Sealant, Perio, Pano, Endo, etc) Profit/Loss is determined by taking the Average Profit/Loss for that group of procedures. By way of example, “endo” stand for endodontics and includes the average profit for all endo procedures in the initial analysis: Anterior, Bicuspid, Molar root canals, etc.

The bottom boxes are where the Profit/Loss for each group of procedures (endo, pano, sealant) are averaged.

In addition, the professional can set his/her discount percentage for each group. That is, he/she can discount all endo procedures by 30%, 20%, or another amount, and the calculation will inform him of his/her averaged Profit/Loss with that discount. In one embodiment, the discount is taken from his/her own fee-for-service fees.

Accordingly, the service provider/professional is able to set up his/her own office service plan with any “promotional yearly premium” for a set of procedures, as well as a specified discount from all other groups of procedures. The service professional can adjust the numbers until he/she gets the desired Profit/Loss.

The following is a representative example:

TABLE 8
Two procedures covered by a promotional (diagnostic/preventative) package of services:

| Procedure 1: Professional's Fee (Fee-For-Service) | Cost of the Service |
|---|---|
| $10 | $5 |
| Procedure 2: Professional's Fee (Fee-For-Service) | Cost of the Service |
| $20 | $10 |
| Total Cost of the Package of Service (Diagnostic/Preventative) | $15 |
| Single Patient/Client Yearly Promotional “Benefit Premium” | Cost of Services Totaled |
| $10 | $15 |

Overall profit or loss for each grouping of services (presuming one service will be provided per profit/loss is the
result of the average for each category and the discount percentage from that profit/loss):
Assuming a Discount of 10% (this can be any amount)
Original Package Only (Diagnostic/Preventative - assuming all are used):
$10 − $15 = −$5 (loss)
Group of Services A:
The Profit/Loss for Group A, Averaged, is $25.
Total Profit/Loss for Package + Group A = $25 + −$5 = $20
Group of Services B:
Profit/Loss for Group B, Average, is $50.
Total Profit/Loss for Package + Group B = $50 + −$5 = $45.
This can be continued for an unlimited group of services.

As mentioned herein, the described systems and methods can be beneficial in several manners. Indeed, in some embodiments, the described systems help a provider to quickly determine which procedures are profitable and which are not. Similarly, some embodiments of the described systems help a provider to recognize which insurance companies are profitable for the provider and which are not. Moreover, in some embodiments, the described systems provide other analytical information that allows the provider to quickly see how the provider's practice can be made more profitable.

Thus, the present invention relates to systems and methods for providing financial analyses to service providers. In particular, some embodiments of the present invention relate to systems and methods for determining the profitability or loss associated with certain procedures and certain insurance companies.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A computer program product for implementing within a computer system a method for obtaining automatic updates on insurance prices to provide a financial analysis of a service provider's practice to reduce inefficiencies in the provider's practice, the computer program product comprising:
a computer-readable, non-transitory medium for providing computer program code means utilized to implement the method, wherein the computer program code means is comprised of executable code for implementing steps comprising:
using a processor of the computer system to gather information, on a continual basis and for a chosen period of time, about a set of chosen procedures performed by the practice, wherein the gathered information includes a reported time required for the provider to perform each of the chosen procedures, the provider's fee for service charge for each of the chosen procedures, dynamic fees that an insurance company is willing to pay for each of the chosen procedures; and a number of each of the chosen procedures that are performed by the provider in the chosen period, wherein at least some of the gathered information is gathered by the computer system on a continual basis via electronic importation;
obtaining the dynamic fees that the insurance company is willing to pay from an automatically updated database; generating, by the processor of the computer system, a weighted percent for each of the chosen procedures by dividing the number of each of the chosen procedures that are performed by the provider in the chosen period by a total number of all of the chosen procedures that are performed by the provider in the chosen period;
generating, by the processor, a net profit/loss for each of the chosen procedures that are performed by the provider in the chosen period;
continually determining, by the processor, a weighted profit/loss for each of the chosen procedures under the fee for service charge and under the dynamic fees that the insurance company is willing to pay by multiplying for each of the chosen procedures, under the fee for service charge and under the dynamic fees that the insurance company is willing to pay, the net profit/loss for each of the chosen procedures performed by the provider in the chosen period by the weighted percent for each of the chosen procedures; and
automatically providing output to reduce the inefficiencies in the provider's practice by controlling selection of at least one of: (i) procedures performed by the provider, (ii) instrumentation and/or materials used by the provider, (iii) payment options accepted by the provider, (iv) procedure pricing, (v) insurance selection, and (vi) billing procedures used by the provider.

2. The computer program product of claim 1, wherein the time required for the provider to perform each of the chosen procedures excludes time spent by personnel of the provider to perform each of the chosen procedures.

3. The computer program product of claim 1, wherein the computer program code means is further comprised of executable code for implementing a step for automatically alerting a user where the provider's fee for service charge for any of the procedures is lower than a corresponding dynamic fee that the insurance company is willing to pay.

4. The computer program product of claim 1, wherein the computer program code means is further comprised of executable code for implementing a step for determining, by the processor, an overall income earned by the provider during the chosen period from the insurance company.

5. The computer program product of claim 1, wherein the gathered information further comprises at least two of: a reported gross income earned by the provider during the chosen period, actual expenses accrued by the provider during the chosen period, a total amount of time worked by the provider during the chosen period, and a gross income earned by the provider during the chosen period from the insurance company.

6. The computer program product of claim 1, wherein the computer program code means is further comprised of executable code for implementing a step for checking, by the processor, the gathered information for correctness by at least one of:
i) determining, by the processor, a freedom time multiplier for each of the chosen procedures by dividing the reported time required for each of the chosen procedures by a calculated time spent on each of the chosen procedures during the chosen period;
ii) determining, by the processor, a factored volume for each of the chosen procedures by multiplying the number of each of the chosen procedures that are reported to have been performed by the provider in the chosen period by an adjusted amount of time that it takes the provider to perform the each of the chosen procedures, wherein the adjusted amount of time is calculated by multiplying the reported time required for the provider to perform each of the chosen procedures by the freedom time multiplier corresponding to each of the chosen procedures; and
iii) determining, by the processor, a total possible income the provider could have earned during the chosen period based on the provider's fee for service charge for each of the chosen procedures and comparing the total possible income against a reported gross income of the provider during the chosen period.

7. The computer program product of claim 5, wherein the computer program code means is further comprised of executable code for implementing a step for identifying, by the processor, a calculated gross income and identifying a potential cause of a discrepancy between the calculated gross income and the reported gross income earned by the provider in during the chosen period.

8. A computer program product for implementing within a computer system a method for obtaining automatic updates on insurance prices to provide a financial analysis of a service provider's practice to reduce inefficiencies in the provider's practice, the computer program product comprising:
a computer-readable, non-transitory medium for providing computer program code means utilized to implement the method, wherein the computer program code means is comprised of executable code for implementing steps comprising:
gathering information, on a continual basis and for a chosen period of time, about a set of chosen procedures performed by the practice, wherein the gathered information includes a reported time required for the provider to perform each of the chosen procedures, the provider's fee for service charge for each of the chosen procedures, dynamic fees that an insurance company is willing to pay for each of the chosen procedures, a number of each of the chosen procedures that are reported to have been performed by the provider in the chosen period, and a reported gross income of the provider during the chosen period, wherein at least some of the gathered information is gathered by the computer system on a continual basis via electronic importation;
obtaining the dynamic fees that the insurance company is willing to pay from an automatically updated database;
checking the gathered information for correctness by at least one of:
i) generating, by a processor of the computer system, a freedom time multiplier for each of the chosen procedures by dividing the reported time required for each of the chosen procedures by a calculated time spent on each of the chosen procedures during the chosen period;
ii) determining, by the processor, a factored volume for each of the chosen procedures by multiplying the number of each of the chosen procedures that are reported to have been performed by the provider in the chosen period by an adjusted amount of time that it takes the provider to perform the each of the chosen procedures, wherein the adjusted amount of time is calculated by multiplying the reported time required for the provider to perform each of the chosen procedures by the freedom time multiplier corresponding to each of the chosen procedures; and
iii) determining, by the processor, a total possible income the provider could have earned during the chosen period based on the provider's fee for service charge for each of the chosen procedures and comparing the total possible income against the reported gross income of the provider during the chosen period;
determining, by the processor, an overall expense amount per hour accrued by the practice;
determining, by the processor, an overall income per hour earned through the insurance company; and
comparing the overall expense per hour to the overall income per hour earned through the insurance company to calculate an hourly profit/loss associated with the insurance company; and
automatically providing output to reduce the inefficiencies in the provider's practice by controlling selection of at least one of: (i) procedures performed by the provider, (ii) instrumentation and/or materials used by the provider, (iii) payment options accepted by the provider, (iv) procedure pricing, and (v) billing procedures used by the provider.

9. The computer program product of claim 8, wherein the computer program code means is further comprised of executable code for implementing a step for automatically alerting a user where the provider's fee for service charge for any of the chosen procedures is lower than a corresponding dynamic fee that the insurance company is willing to pay.

10. The computer program product of claim 8, wherein the time required for the provider to perform each of the chosen procedures excludes time spent by personnel of the provider to perform each of the chosen procedures.

11. The computer program product of claim 8, wherein the reported gross income of the provider during the chosen period excludes personal cash management income.

12. The computer program product of claim 8, wherein the computer program code means is further comprised of executable code for implementing a step for identifying a calculated gross income and identifying a potential cause of a discrepancy between the calculated gross income and the reported gross income earned by the provider in the chosen period.

13. The computer program product of claim 8, wherein the step of checking the gathered information for correctness comprises at least one of: calculating, by the processor, a total expense for each of the chosen procedures during the chosen period, calculating, by the processor, a revenue earned during the chosen period for each of the chosen procedures, calculating, by the processor, a total possible income earned during the chosen period by the provider for each of the chosen procedures, and calculating, by the processor, an adjusted net un-weighted profit/loss for each of the chosen procedures during the chosen period.

14. A computer program product for implementing within a computer system a method for obtaining automatic updates on insurance prices to provide a financial analysis of a service provider's practice to reduce inefficiencies in the provider's practice, the computer program product comprising:
a computer-readable, non-transitory medium for providing computer program code means utilized to implement the method, wherein the computer program code means is comprised of executable code for implementing steps comprising:
gathering information, on a continual basis and for a chosen period of time, about a subset of chosen procedures selected from all procedures performed by the practice, wherein the gathered information includes a reported time required for the provider, and not the provider's support personnel, to perform each of the chosen procedures, the provider's fee for service charge for each of the chosen procedures; dynamic fees that an insurance company is willing to pay for each of the chosen procedures; and a number of each of the chosen procedures that are performed by the provider in the chosen period, wherein at least some of the gathered information is gathered by the computer system on a continual basis via electronic importation;
obtaining the dynamic fees that the insurance company is willing to pay from an automatically updated database;
generating, by a processor of the computer system, a weighted percent for each of the chosen procedures by dividing the number of each of the chosen procedures that are performed by the provider in the chosen period by a total number of all of the chosen procedures that are performed by the provider in the chosen period;
generating, by the processor, a net profit/loss for each of the chosen procedures that are performed by the provider in the chosen period;
continually determining, by the processor, a weighted profit/loss for each of the chosen procedures under the fee for service charge and under the dynamic fees that the insurance company is willing to pay by multiplying for each of the chosen procedures, under the fee for service charge and under the dynamic fees that the insurance company is willing to pay, the net profit/loss for each of the chosen procedures performed by the provider in the chosen period by the weighted percent of each of the chosen procedures; and
automatically providing output to reduce the inefficiencies in the provider's practice by controlling selection of at least one of: (i) procedures performed by the provider, (ii) instrumentation and/or materials used by the provider, (iii) payment options accepted by the provider, (iv) procedure pricing, (v) insurance selection, and (iv) billing procedures used by the provider.

15. The computer program product of claim 14, wherein the gathered information further comprises at least two of: a reported gross income earned by the provider during the chosen period, actual expenses accrued by the provider during the chosen period, a total amount of time worked by the provider during the chosen period, and a gross income earned by the provider during the chosen period from the insurance company.

16. The computer program product of claim 14, wherein the computer program code means is further comprised of executable code for implementing a step for automatically alerting a user where the provider's fee for service charge for any of the procedures is lower than a corresponding dynamic fee that the insurance company is willing to pay.

17. The computer program product of claim 15, wherein the computer program code means is further comprised of executable code for implementing a step for identifying, by the processor, a calculated gross income and identifying a potential cause of a discrepancy between the calculated gross income and the reported gross income earned by the provider in during the chosen period.

18. The computer program product of claim 14, wherein the computer program code means is further comprised of executable code for implementing a step for checking the information for correctness by at least one of:
i) determining, by the processor, a freedom time multiplier for each of the chosen procedures by dividing the reported time required for each of the chosen procedures by a calculated time spent on each of the chosen procedures during the chosen period;
ii) determining, by the processor, a factored volume for each of the chosen procedures by multiplying the number of each of the chosen procedures that are reported to have been performed by the provider in the chosen period by an adjusted amount of time that it takes the provider to perform the each of the chosen procedures, wherein the adjusted amount of time is calculated by multiplying the reported time required for the provider to perform each of the chosen procedures by the freedom time multiplier corresponding to each the chosen procedures; and
iii) determining, by the processor, a total possible income the provider could have earned during the chosen period based on the provider's fee for service charge for each of the chosen procedures and comparing the total possible income against a reported gross income of the provider during the chosen period.
